(19) United States
(12) Patent Application Publication
(10) Pub. No.: US 2005/0063969 A1
(43) Pub. Date: Mar. 24, 2005

(54) Assay

(76) Inventors: Simon Barry, Stevenage (GB); Carmel Horgan, Stevenage (GB); Steven Ludbrook, Stevenage (GB); David Miller, Stevenage (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/471,814

(22) PCT Filed: Mar. 14, 2002
(86) PCT No.: PCT/GB02/01212

(30) Foreign Application Priority Data
Mar. 14, 2001 (GB) 0106278.5

(51) Int. Cl. G01N 33/53; G01N 33/567; A61K 39/395

(52) U.S. Cl. 424/144.1; 435/7.2

(57) ABSTRACT

A method for the identification of a modular of the interaction between latency associated peptide (LAP) of transforming growth factor β3 (TGF-β3) and αv integrin, which method comprises: (a) providing, as a first component, LAP-β3 or a functional variant thereof; (b) providing, as a second component, and αv integrin or a functional variant thereof; (c) contacting the two components with a test product under conditions that, in the absence of the test product, would permit the two components to interact; and (d) determining whether the test product is capable of modulating the interaction between the first and second components, thereby to determine whether the test product is a modulator of the interaction between LAP-β3 and the integrin.

### FIELD OF THE INVENTION

The invention relates to medical products for use in immunomodulation and use in the treatment of inflammatory and fibrotic diseases and cancer, diabetic retinopathy, bone resorption and osteoporosis. The invention also relates to methods for identifying products which are useful in therapy, particularly in immunomodulation and in the treatment of inflammatory and fibrotic diseases and fibrotic diseases and cancer, diabetic retinopathy, bone resorption and osteoporosis.

### BACKGROUND TO THE INVENTION

The integrins comprise a large family of heterodimeric transmembrane receptors that mediate both cell-cell and cell-matrix interactions. They engage numerous ligands and regulate a variety of cellular and physiological processes such as cell proliferation, apoptosis, migration, differentiation, inflammation, and tissue remodelling.

The αᵥ integrin family comprises the integrins αᵥβ₁, αᵥβ₃, αᵥβ₅, αᵥβ₆ and αᵥβ₈. The family members are expressed on a wide variety of cells with one or more members being expressed on virtually all cell types. For example, αᵥβ₃ has been shown to be expressed on endothelial cells, smooth muscle cells, a variety of monocyte-derived cells, tumor cells, platelets, mesenchymal fibroblasts, T lymphocytes, and dendritic cells. αᵥβ₆ appears to have a more restricted distribution, being present on epithelial cells, tumor cells, and possibly some inflammatory cell types.

The transforming growth factor-β (TGF-β) isoforms TGF-β1, TGF-β2 and TGF-β3 are expressed from numerous cell types and are present in virtually all tissues. They potently inhibit cellular proliferation of many cell types, and also induce extracellular matrix synthesis, integrin expression, and modulate immune responses. They are synthesised as large precursor proteins that are proteolytically processed in the golgi to yield a mature TGF-β protein of approximately 110 amino acids and an amino terminal protein called latency associated protein-β (LAP-β) of approximately 280 amino acids. These two proteins homodimerise and the two dimers also interact to form a LAP-β-TGF-β complex containing two parts of each protein. This complex renders TGF-β inactive, and activation requires some conformational change and/or proteolysis of the LAP-β protein.

### SUMMARY OF THE INVENTION

This invention is based on our finding that the αᵥ family of integrins bind the latency associated peptide-β3 (LAP-β3) of transforming growth factor-β3 (TGF-β3). The interaction between these integrins and LAP-β3 of TGF-β3 provides a link between the interaction and a number of biologically significant processes.

According to the present invention there is thus provided a method for the identification of a modulator of the interaction between latency associated peptide-β3 and an αᵥ integrin, which method comprises:
(a) providing, as a first component, latency-associated peptide-β3 (LAP-β3) or a functional variant thereof;
(b) providing, as a second component, an αᵥ integrin or a functional variant thereof;
(c) contacting the two components with a test product under conditions that, in the absence of the test product, would permit the two components to interact; and
(d) determining whether the test product is capable of modulating the interaction between the first and second components,
   thereby to determine whether the test product is a modulator of the interaction between LAP-β3 and the αᵥ integrin.

The method of the invention may also be used to identify modulators of TGF-β3 activity.

The invention also provides:
a test kit for the identification of a modulator of the interaction between LAP-β3 and an αᵥ integrin, which kit comprises:
   (i) a first component which is latency-associated peptide-β3 (LAP-β3) or a functional variant thereof; and
   (ii) a second component which is, an αᵥ integrin or a functional variant thereof;
a modulator of the interaction between LAP-β3 and an αᵥ integrin for use in a method of treatment of the human or animal body;
use of a modulator which is an inhibitor of the interaction between LAP-β3 and an αᵥ integrin in the manufacture of a medicament for use in a method of immuno-modulation or in a method of treatment of an inflammatory disease, a fibrotic disease, cancer, diabetic retinopathy, bone resorption or osteoporosis;
use of a modulator which is an activator of the interaction between LAP-β3 and an αᵥ integrin in the manufacture of a medicament for use in a method of preventing apoptosis;
a pharmaceutical composition comprising a pharmaceutically acceptable carrier or diluent and, as active ingredient, a product which is a modulator of the interaction between LAP-β3 and an αᵥ integrin;
a method for treating a host requiring immuno-modulation or suffering from an inflammatory disease, a fibrotic disease, cancer, diabetic retinopathy, bone resorption or osteoporosis which method comprises administering to the host an effective amount of a product which is an inhibitor of the interaction between LAP-β3 and an αᵥ integrin;
a method for treating a host requiring prevention of apoptosis which method comprises administering to the host an effective amount of an activator of the interaction between LAP-β3 and an αᵥ integrin;
a modulator of the interaction between LAP-β3 and an αᵥ integrin identified by a method of the invention or a test kit of the invention;
a modulator of the interaction between LAP-β3 and an αᵥ integrin identified by a method of the invention or a test kit of the invention for use in a method of treatment of the human or animal body by therapy;
use of a modulator of the interaction between LAP-β3 and an αᵥ integrin identified by a method of the invention or a test kit of the invention in the manufacture of a medicament for use in a method of immuno-modulation or a method of treatment of an inflammatory disease, a fibrotic disease, cancer, diabetic retinopathy, bone resorption or osteoporosis;
use of a modulator of the interaction between LAP-β3 and an αᵥ integrin identified by a method of the invention or a test kit of the invention in the manufacture of a medicament for use in a method of preventing apoptosis;
a pharmaceutical composition comprising a pharmaceutically acceptable carrier or diluent and, as active ingredient, a modulator of the interaction between LAP-β3 and an αᵥ integrin identified by a method of the invention or a test kit of the invention;
a method for treating a host requiring immuno-modulation or suffering from an inflammatory disease, a fibrotic disease, cancer, diabetic retinopathy, bone resorption or osteoporosis, which method comprises administering to the host an effective amount of a modulator of the interaction between LAP-β3 and an αᵥ integrin identified by a method of the invention or a test kit of the invention;
a method for treating a host requiring prevention of apoptosis, which method comprises administering to the host an effective amount of a modulator of the interaction between LAP-β3 and an αᵥ integrin identified by a method of the invention or a test kit of the invention;
a method for identifying a product which can be used in a method of immuno-modulation or in a method of treatment of an inflammatory disease, a fibrotic disease, cancer, diabetic retinopathy, bone resorption or osteoporosis, which method comprises:
   (a) providing, as a first component, latency-associated peptide-β3 (LAP-β3) or a functional variant thereof;
   (b) providing, as a second component, an αᵥ integrin or a functional variant thereof;
   (c) contacting the two components with a test product under conditions that, in the absence of the test product, would permit the two components to interact;
   (d) determining whether the test product is capable of inhibiting the interaction between the first and second components; and
   (e) determining whether a product identified in step (d) can be used in a method of immuno-modulation or in the treatment of an inflammatory disease, a fibrotic disease, cancer, diabetic retinopathy, bone resorption or osteoporosis;
a method for identifying a product which can be used in a method of preventing apoptosis, which method comprises:
   (a) providing, as a first component, latency-associated peptide-β3 (LAP-β3) or a functional variant thereof;
   (b) providing, as a second component, an αᵥ integrin or a functional variant of a functional variant thereof;
   (c) contacting the two components with a test product under conditions that, in the absence of the test product, would permit the two components to interact;
   (d) determining whether the test product is capable of activating the interaction between the first and second components; and
   (e) determining whether a product identified in step (d) can be used in a method of preventing apoptosis;
a method for treating a host requiring immuno-modulation or suffering from an inflammatory disease, a fibrotic disease, cancer, diabetic retinopathy, bone resorption or osteoporosis, which method comprises:
   (a) identifying a product by use of a method for identifying a product which can be used in a method of immuno-modulation or in a method of treatment of an inflammatory disease, a fibrotic disease, cancer, diabetic retinopathy, bone resorption or osteoporosis, which method comprises; and
   (b) administering to the host an effective amount of the product; and
a method for treating a host requiring prevention of apoptosis, which method comprises:
   (a) identifying a product by use of a method for identifying a product which can be used in a method of preventing apoptosis; and
   (b) administering to the host an effective amount of the product.

### BRIEF DESCRIPTION OF THE FIGURES

FIG. 1 shows the adhesion of K562-αᵥβ₃, K562-αᵥβ₅, and K562-αᵥβ₆ cells to wells coated with 0.5 μg of a fusion protein of GST and amino acids 259-269 of LAPβ₃ in the presence of either 1 mM EDTA, 2 mM MgCl₂, or 0.5 mM MnCl₂, as indicated under the bars. Each data point represents the mean±SD of duplicate points, and is representative of at least three identical experiments.

FIG. 2 shows the specificity of K562-αᵥβ₃ cell adhesion to GST-LAPβ₃ (amino acids 259-269). K562-αᵥβ₃ cells were allowed to attach to wells coated with 0.5 μg of GST-LAPβ₃ (amino acids 259-269) in the presence of 2 mM MgCl₂. Specific blocking antibodies (1 μg/well) and compounds were added as indicated. Each data point represents the mean±SD of duplicate points, and is representative of at least three identical experiments.

FIG. 3 shows the specificity of K562-αᵥβ₆ cell adhesion to GST-LAPβ₃ (amino acids 259-269). K562-αᵥβ₆ cells were allowed to attach to wells coated with 0.5 μg of GST-LAPβ₃ (amino-acids 259-269) in the presence of 2 mM MgCl₂. Specific blocking antibodies (1 μg/well) and compounds were added as indicated. Each data point represents the mean±SD of duplicate points, and is representative of at least three identical experiments.

FIG. 4 shows the specificity of K562-αᵥβ₅ cell adhesion to GST-LAPβ₃ (amino acids 259-269). K562-αᵥβ₅ cells were allowed to attach to wells coated with 0.5 μg of GST-LAPμ₃ (amino acids 259-269) in the presence of 0.5 mM MnCl₂. Specific blocking antibodies (1 μg/well) and compounds were added as indicated. Each data point represents the mean±SD of duplicate points, and is representative of at least three identical experiments.

FIG. 5 shows the importance of the LAPβ₃ RGD sequence for K562-αᵥβ₃, K562-αᵥβ₅ and K562-αᵥβ₆ cell binding. K562-αᵥβ₃, K562-αᵥβ₅, and K562-αᵥβ₆ cells were allowed to attach to wells coated with 0.5 μg of either GST-LAPβ₃ (amino acids 259-269), or site specific mutants where either Arg₂₆₁, Gly₂₆₂, or AsP₂₆₃ were mutated to alanine as described under the bars. Adhesion occurred in the presence of 2 mM MgCl₂ for K562-αᵥβ₃ and K562-αᵥβ₆ cells or 0.5 mM MnCl₂ for K562-αᵥβ₅ cells. Each data point represents the mean±SD of duplicate points, and is representative of at least three identical experiments.

### DESCRIPTION OF THE SEQUENCE LISTING

SEQ ID NO: 1 (SwissPot Accession No. P10600) sets out the amino acid sequence of human TGF-β3. Amino acids 1 to 20 is the signal sequence; amino acids 21 to 300 is the LAP-β3 sequence; and amino acids 301 to 412 is the mature TFG-β3 cytokine sequence.

### DETAILED DESCRIPTION OF THE INVENTION

We have shown, surprisingly, that αᵥ integrins bind to the latency associated peptide (LAP-β3) region of transforming growth factor-β3 (TGF-β3). This links the interaction of the latency associated peptide (LAP-β3) with αᵥ integrins with the following biological processes:
(a) activation of TGF-β3 and subsequent immunomodulation and/or fibrosis mediated by active TGF-β3;
(b) migration of cells expressing an αᵥ integrin along LAP-TGFβ3 protein; and
(c) protection of cells expressing an αᵥ integrin from apoptosis.

Due to the presence of αᵥ integrins on a wide variety of cell types, and the presence of TGF-β3 in many tissues, originating from many cell types, there may be multiple effects of the interaction of these integrins with LAP-β3 of TGF-β3. Therefore, all cell types that express one or more αᵥ integrin have the potential to interact with LAP-β3. This may modulate cell activation/differentiation via the binding event to the integrin (i.e. signalling through the integrin) or via the activation of TGFβ3 (i.e. TGF-β3 signalling through TGFβ receptors), and the immunomodulatory (including Th1/Th2 cell switching)/fibrotic effects of activated TGF-β3.

The invention provides methods for identifying a modulator of the interaction between LAP-β3 and an αᵥ integrin. A suitable method of the invention comprises: providing, as a first component, LAP-β3 or a functional variant thereof; providing, as a second component an αᵥ integrin or a functional variant thereof; contacting the two components with a test product under conditions that, in the absence of the test product, would permit the two components to interact; and determining whether the test product is capable of inhibiting the interaction between the first and second components. The skilled person can thereby readily determine whether the test product is a modulator of the interaction between LAP-β3 and an αᵥ integrin.

In vivo, TGF-β3 is secreted as a complex composed of three proteins derived from two genes. The TGF-β3 gene encodes a procytokine consisting of a C-terminal TGF-β3 sequence and a larger N-terminal region that after processing forms the latency-associated peptide-β3 (LAP-β3). Both the mature TGF-β3 arid LAP-β3 peptides form homodimers and the two homodimers form a noncovalent complex called the small latent complex (SLC). LAP-β3 can disulfide bond to another protein, the latent TGF-β3-binding protein (LTBP); the latent form of TGF-β3 thus formed is called the large latent complex (LLC).

LAP-β3 or a functional variant thereof is provided as a first component. The amino acid sequence of LAP-β3 is set out as amino acids 21 to 300 of SEQ ID NO:1. The sequence of SEQ ID NO: 1 corresponds to that of SwissProt database accession number P10600. LAP-β3 may be used as a monomer, but is more typically used as a homodimer. If a homodimer is used one or both peptides comprising the dimer may be a functional variant of LAP-β3. If both peptides are functional variants they may be different functional variants of LAP-β3.

A functional variant of LAP-β3 is a polypeptide which has a sequence similar to that of amino acids 21 to 300 of SEQ ID NO: 1 and which retains the ability to bind an αᵥ integrin. Typically, the binding activity to the integrin of a functional variant may be substantially the same as that of LAP-β3. Alternatively, the binding activity of a functional variant may be greater or less than that of LAP-β3. For example, a functional variant may have at least 90% activity, at least 80% activity or at least 70% activity of the LAP-β3 having the sequence set out as amino acids 30 to 278 of SEQ ID NO: 1 with respect to its ability to bind an αᵥ integrin.

A functional variant typically comprises a sequence substantially similar to that set out as amino acids 21 to 300 of SEQ ID NO: 1. Thus a functional variant will generally have at least 60%, at least 70%, at least 80%, at least 90%, at least 95%, at least 98 or at least 99% sequence identity to the LAP-β3 having the sequence set out as amino acids 21 to 300 of SEQ ID NO: 1, calculated over the fulll length 6f those sequences. The UWGCG Package provides the BESTFIT program which can be used to calculate identity (for example used on its default settings) (Devereux et al (1984) Nucleic Acids Research 12; β387-395). The PILEUP and BLAST algorithms can be used to calculate identity or line up sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Centre for Biotechnology Information (http://www.ncbi.nlm.nih.gov/).

A functional variant may be a naturally occurring sequence, such as a related gene, for example LAP-β1, or an allelic variant of LAP-β3. An allelic variant will generally be of human or non-human mammal, for example bovine or porcine, origin.

Alternatively, a functional variant may be a non-naturally occurring sequence. A non-naturally occurring functional variant may be a modified version of the LAP-β3 having the sequence set out as amino acids 21 to 300 of SEQ ID NO: 1, obtained by, for example, amino acid substitution, deletion or addition. Up to 1, up to 5, up to 10, up to 50 or up to 100 amino acid substitutions or deletions, for example, may be made. Thus, a functional variant of the sequence given as amino acids 21 to 300 of SEQ ID NO: 1 may be a fragment of that sequence. Typically, if substitutions are made, the substitutions will be conservative substitutions, for example according to the following Table. Amino acids in the same block in the second column and preferably in the same line in the third column may be substituted for each other. Deletions are preferably deletions of amino acids from one or both ends of the sequence given as amino acids 21 to 300 of SEQ ID NO: 1. Alternatively, deletions are of regions not involved in the binding of an αᵥ integrin.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Preferred polypeptides comprise the ROD motif at amino acids 261 to 263 of SEQ ID NO: 1, such as a fragment comprising amino acids 259 to 269 of SEQ ID NO: 1.

LAP-β3 or a functional variant thereof may be fused to a carrier polypeptide. Thus, additional amino acid residues may be provided at, for example, one or both termini of LAP-β3 or a functional variant thereof for the purpose of providing a carrier polypeptide, by which the polypeptide can be, for example, affixed to a label, solid matrix or carrier. Thus the first component for use in a method of the invention may be in the form of a fusion polypeptide which comprises heterologous sequences. Indeed, in practice it may often be convenient to use fusion polypeptides. This is because fusion polypeptides may be easily and cheaply produced in recombinant cell lines, for example recombinant bacterial or insect cell lines. In addition, fusion polypeptides may be easy to identify and isolate. Typically, fusion polypeptides will comprise a polypeptide sequence as described above and a carrier or linker sequence. The carrier or linker sequence will typically be derived from a non-human, preferably a non-mammalian source, for example a bacterial source. This is to minimize the occurrence of non-specific interactions between sequences in the fusion polypeptide and the integrin.

Polypeptides may be modified by, for example, addition of histidine residues, a T7 tag or glutathione S-transferase, to assist in their isolation. Alternatively, the carrier polypeptide may, for example, promote secretion of the polypeptide from a cell or target expression of the polypeptide to the cell membrane. Amino acids carriers can be from 1 to 400 amino acids in length or more typically from 5 to 200 residues in length. The polypeptide may be linked to a carrier polypeptide directly or via an intervening linker sequence. Typical amino acid residues used for linking are tyrosine, cysteine, lysine, glutamic acid or aspartic acid.

A polypeptide comprising LAP-β3 or a functional variant thereof may be provided in association with the processed TGF-β3 propeptide or a functional variant thereof That is to say, a small latent complex (SLC) can be used in the invention. Functional variants of the processed TGF-β3 propeptide retain LAP-β3 binding activity and may be obtained according to the description provided above for LAP-β3.

A polypeptide comprising LAP-β3 or a functional variant thereof may be provided in association with the processed TGF-β3 propeptide or a functional variant thereof, and the latent TGF-β3-binding protein (LTBP) or a functional variant thereof. That is to say, a large latent complex (LLC) can be used in the invention. Functional variants of the LTBP retain LAP-β3 binding activity and may be obtained according to the description provided above for LAP-β3.

Suitable polypeptides for use as a first component may be chemically modified, for example, post translationally modified. For example they may be glycosylated or comprise modified amino acid residues. Polypeptides can be in a variety of forms of polypeptide derivatives, including amides and conjugates with polypeptides i.e. LAP-β3 and/or processed TGF-β3 and/or LTBP or functional variants thereof may be so-modified.

Chemically modified polypeptides also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

Also included as chemically modified polypeptides are those polypeptides which contain one or more naturally occurring amino acids derivatives of the twenty standard amino acids. For example, 4-hydroxyproline may be substituted for proline or homoserine may be substituted for serine.

LAP-β3 or a functional variant thereof and/or other polypeptides used as part of a first component may carry a revealing label. Suitable labels include radioisotopes such as ³²p or ³⁵S, fluorescent labels, enzyme labels, or other protein labels such as biotin.

LAP-β3 or a functional variant thereof and/or other polypeptides used as part of a first component may be expressed using recombinant DNA techniques. For example, suitable polypeptides may be expressed in, for example, bacterial or insect cell lines (see, for example, Munger et al., 1998, Molecular Biology of the Cell, 9, 2627-2638). Also, suitable polypeptides may be isolated biochemically from any suitable tissue.

Alternatively, polypeptides may be chemically synthesized. Synthetic techniques, such as a solid-phase Merrifield-type synthesis, may be preferred for reasons of purity, antigenic specificity, freedom from unwanted side products and ease of production. Suitable techniques for solid-phase peptide synthesis are well known to those skilled in the art (see for example, Merrifield et al., 1969, Adv. Enzymol 32, 221-96 and Fields et al., 1990, Int. J. Peptide Protein Res, 35, 161-214). In general, solid-phase synthesis methods comprise the sequential addition of one or more amino acid residues or suitably protected amino acid residues to a growing peptide chain.

Polypeptides for use as a first component in a method of the invention may be linear or cyclic. A linear polypeptide may be cyclised according to any suitable method (see for example Zimmer et al., 1992, Peptides, pp.393-394, ESCOM Science Publishers, BV., 1993 and Gurrath et al., 1992, Eur. J. Biochem., 210, 911-921). Typically, tertbutoxycarbonyl protected polypeptide methyl ester is dissolved in methanol and sodium hydroxide are added and the admixture is reacted at 20° C. to hydrolytically remove the methyl ester protecting group. After evaporating the solvent, the tertbutoxycarbonyl protecting group is extracted with ethyl acetate from acidified aqueous solvent. The tertbutoxycarbonyl protecting group is then removed under mildly acidic conditions in dioxane cosolvent. The unprotected linear peptide with free amino acid and carboxy termini so obtained is converted to its corresponding cyclic polypeptide by reacting a dilute solution of the linear polypeptide in a mixture of dimethylformamide with dicyclohexylcarboiimide in the presence of 1-hydroxy benzotriazole and N-methylmorpholine. The resultant cyclic polypeptide is purified by chromatography.

The second component comprises an integrin of the αᵥ integrin family or a functional variant thereof. The αᵥ family of integrins comprises αᵥβ₁, αᵥβ₃, αᵥβ₅, αᵥβ₆ and αᵥβ₈ integrin and any of these, or a functional variant, may be the second component or the integrin employed in the invention. In a preferred embodiment of the invention the second component is αᵥβ₃ or αᵥβ₆ integrin or a functional variant of either.

A functional variant of one of these integrins is a polypeptide which shows an activity like one of the αᵥ integrins in respect of the ability of the integrin to bind TGF-β3. More specifically, the functional variant is able to bind LAP-β3 of TGF-β3. Thus, the second component may comprise, for example, a fragment of an αᵥ integrin which binds LAP-β3, or a polypeptide which comprises the wild type sequences of an αᵥ integrin which correspond to LAP-β3 binding sites and elsewhere comprises non-wild type sequences.

Also, suitable functional variants may comprise non-wild type LAP-β3 binding sites, but will still be capable of binding LAP-β3. It may be preferable to use non-wild type binding sites which show an increased binding affinity for LAP-β3 as compared with the binding affinity of wild type binding sites. Use of such non-wild type binding sites may allow the identification of products which are strong disrupters of interactions between LAP-β3 and an αᵥ integrin. Non-wild type LAP-β3 binding sequences will typically arise through substitution, deletion or addition for example, as described for the first component above.

Second component polypeptides may be produced according to similar methods as described for first component polypeptides.

A two component assay can be carried out according to any suitable protocol. Preferably, the assay is adapted so that it can be carried out in a single reaction vessel and more preferably can be carried out in a single well of a plastics microtitre plate and thus can be adapted for high through-put screening. Typically, a cell adhesion assay is carried out.

In a cell adhesion assay, the first component polypeptide is coated on the walls of a suitable vessel, in particular the well of a plastic microtitre plate. In one suitable assay format, the second component, produced, for example, chemically or recombinantly is simply added to the assay vessel. Binding of the second component to the first component can be followed by the use of a second component which carries a label, for example a radioactive label or a fluorescent label.

Alternatively, in another suitable assay format, cells expressing the second component are added to the, vessel and allowed to interact with the first component in the presence of a test product. Suitable cells are any cells that express one or more integrin. Numerous cells and cell lines, both primary and transformed, express one or more αᵥ integrin and may be used in the assay. These include, for example, Dx3 melanoma cells for αᵥβ₁, αᵥβ₃ and αᵥβ₅, HT29 cells for αᵥβ₅ and αᵥβ₆ and SW480 cells for αᵥβ₅. Alternatively integrin cDNAs in expression vectors can be transfected into cells to get suitable cells for use in the assays, as described for example in Blystone et al (1995) Journal of Cell Biology 130: 745-754.

The number of cells which bind to the first component polypeptide is then determined. This may be carried out by, for example, staining the cells and then carrying out spectrophotometry. Optionally, the stain may be eluted and spectrophotometry carried out on the eluted sample.

It may be necessary to add further components to the reaction mixture in order to promote the αᵥ integrin to a suitable activation state for binding to LAP-β3. In addition, suitable control experiments may be carried out. The cell adhesion assay may be run without the test product present. In order to distinguish between non-specific interactions between the first component and cells expressing the second component, antibodies specific to one of the two polypeptides of the integrin may be added to the reaction mixture. Control cells expressing polypeptides other than the second component may be used, to distinguish between specific reactions between the first and second components and non-specific reactions between the αᵥ integrin and other surface proteins of the cells expressing the second component.

The invention also provides a test kit for the identification of a modulator of the interaction of LAP-β3 and an αᵥ integrin. A kit according to the invention comprises a first component as described above and a second component as described above. In a preferred kit of the invention, the kit also comprises a means for determining whether a test product modulates the interaction between the first and second components. Typically, a test kit will provide suitable components for carrying out an adhesion assay as described above. Thus, the second component may be provided in the form of cells which express the second component. If the kit is an adhesion assay kit, the kit may also comprise a suitable stain for quantifying the amount of cells which bind to the first component, for example crystal violet stain.

A kit of the invention may optionally further comprise, appropriate buffer(s), control cells, expressing for example integrins other than αᵥ integrins or control antibodies. A kit of the invention may also comprise appropriate packaging and instructions for use in a method for the identification of a modulator of the interaction between LAP-β3 and the integrin.

Suitable test products for use in methods of the invention include combinatorial libraries, defined chemical entities, peptides and peptide mimetics, oligonucleotides and natural product libraries. Test products may be used in an initial screen of, for example, ten substances per reaction, and the products of batches which show inhibition tested individually. Furthermore, antibody products (for example, monoclonal and polyclonal antibodies, single chain antibodies, chimaeric antibodies, CDR-grafted antibodies and humanized antibodies) may be used.

A modulator of the interaction between LAP-β3 and an αᵥ integrin is one which produces a measurable reduction or increase in the degree of interaction between those two proteins in a method of the invention. Thus, modulators of the interaction between LAP-β3 and the αᵥ integrin may be inhibitors or activators of that interaction.

An inhibitor of the interaction between LAP-β3 and an αᵥ integrin is one which causes the degree of interaction between LAP-β3 and the integrin to be reduced or substantially eliminated, as compared to the degree of interaction between the two, in the absence of that inhibitor. Preferred inhibitors are those which inhibit the interaction between LAP-β3 and the αᵥ integrin by at least 10%, at least 20%, at least 30%, at least 40%, at least 50%, at least 60%, at least 70%, at least 80%, at least 90%, at least 95% or at least 99% at a concentration of the inhibitor of 1 μgml⁻¹, 10 μgml⁻¹, 100 μgml⁻¹, 500 μgml⁻¹, 1 mgml⁻¹, 10 mgml⁻¹, 100 mg ml⁻¹. The percentage inhibition represents the percentage decrease in the interaction between LAP-β3 and the αᵥ integrin in a comparison of assays in the presence and absence of the test substance. Any combination of the above mentioned degrees of percentage inhibition and concentration of inhibitor may be used to define an inhibitor of the invention, with greater inhibition at lower concentrations being preferred.

An activator of the interaction between LAP-β3 and an αᵥ integrin is one which causes the degree of interaction between LAP-β3 and the integrin to be increased, as compared to the degree of interaction between the two in the absence of that activator. Preferred activators are those which activate the interaction between LAP-β3 and the αᵥ integrin by at least 10%, at least 25%, at least 50%, at least 100%, at least, 200%, at least 500o or at least 1000% at a concentration of the activator of 1 μg ml⁻¹, 10 μg ml⁻¹, 100 μg ml⁻¹, 500 μg ml⁻¹, 1 mg ml⁻¹, 10 mg ml⁻¹, 100 mg ml⁻¹. The percentage activation represents the percentage increase in the interaction between LAP-β3 and the αᵥ integrin in a comparison of assays in the presence and absence of the test substance. Any combination of the above mentioned degrees of percentage activation and concentration of activator may be used to define an activator of the invention, with greater activation at lower concentrations being preferred.

Test products which show activity in assays such as those described above can be tested in in vivo systems, such as an animal disease model. Thus, candidate inhibitors could be tested for their ability to attenuate inflammation and/or fibrosis in mice. A candidate activator could be tested for its ability to prevent apoptosis in mice.

It is preferable that inhibitors or activators of the interaction between LAP-β3 and the αᵥ integrin do not disrupt the interaction between LAP-β3 and other integrins such as non αᵥ integrins. That is, inhibitors or activators are generally specific for the interaction between LAP-β3 and an αᵥ integrin.

Suitable inhibitors may include functional variants of an αᵥ integrin, fragments thereof, mimetics of any of these integrins or a natural ligand of these integrins, for example polypeptides based on TGF-β3 that mimic the structural region involved in the binding interactions between LAP-β3 and the αᵥ integrin, polypeptides having a sequence corresponding to a functional binding domain of LAP-β3 for the integrin and antibodies which immunoreact with either LAP-β3 or the integrin.

Suitable activators include functional variants of LAP-β1 or LAP-β3.

Modulators of the invention may be in substantially purified form. They may be in substantially isolated form, in which case they will generally comprise at least 80% e.g. at least 90, 95, 97 or 99% by weight of the dry mass in the preparation. The product is typically substantially free of other cellular components. The product may be used in such a substantially isolated, purified or free form in the method or be present in such forms in a kit.

Modulators of the invention may be used in a method of treatment of the human or animal body by therapy.

In particular, inhibitors of the invention may be use in a method of immuno-modulation or in a method of treatment of an inflammatory disease, a fibrotic disease, cancer (including solid tumor therapy and metastatic tumor therapy), diabetic retinopathy, bone resorption or osteoporosis. Examples of conditions which involve an inflammatory and/or fibrotic component are chronic obstructive pulmonary disorder, rheumatoid arthritis, psoriasis, restenosis, atherosclerosis, liver fibrosis and asthma. The condition of a patient requiring an inhibitor of the interaction between LAP-β3 and an αᵥ integrin can be improved by administration of an inhibitor of the invention. A therapeutically effective amount of an inhibitor of the invention may be given to a host in need thereof.

Activators of the invention may be used in a method of preventing (i.e. protection against) apoptosis. The condition of a patient requiring an activator of the interaction between LAP-β3 and an αᵥ integrin can be improved by administration of an activator of the invention. A therapeutically effective amount of an activator of the invention may be given to a host in need thereof.

Modulators of the interaction between LAP-β3 and an αᵥ integrin may be administered in a variety of dosage forms. Thus, they can be administered orally, for example as tablets, troches, lozenges, aqueous or oily suspensions, dispersible powders or granules. The modulators may also be administered parenterally, either subcutaneously, intravenously, intramuscularly, intrasternally, transdermally or by infusion techniques. The modulators may also be administered as suppositories. A physician will be able to determine the required route of administration for each particular patient.

The formulation of a modulator for use in preventing or treating one of the above-mentioned conditions will depend upon factors such as the nature of the exact modulator, whether a pharmaceutical or veterinary use is intended, etc. A modulator may be formulated for simultaneous, separate or sequential use.

A modulator is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of a modulator is administered to a patient. The dose of a modulator may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

The following Examples illustrate the invention:

### EXAMPLES

Materials & Methods

Unless indicated otherwise, the techniques and methodologies described are standard biochemical techniques. Examples of suitable general methodology textbooks include Sambrook et al., Molecular Cloning (1995), John Wiley & Sons, Inc.

Cell Culture

K562-WT (wild-type), K562-αᵥβ₆, K562-αᵥβ₅ and K562-αᵥβ₃ cells were maintained in 1:1 RPMI 1640, Hepes modification (Gibco): Dulbecco's Minimum Essential Medium (DMEM), Hepes modification (Sigma) supplemented with L-glutamine (Gibco) and 10% fetal calf serum (FCS). In addition K562-αᵥβ₃, K562-αᵥβ₅, and K562-αᵥβ₆ cells were supplemented with geneticin (G418, Gibco) at 1 mg/ml. All transfected cells were generated by transfecting K562 cells with pCDNA-3 constructs containing individual integrin cDNAs, using lipofectamine plus (Gibco BRL). Stable G418-resistant populations were shown to express the required integrin and possess the expected adhesion properties of that integrin.

Antibodies and Other Reagents

Antibody clones used were as follows. The anti-β₁ integrin antibody clone 4B4 was obtained from Coulter. The anti-α₅ antibody clone SAM-1, and anti-αᵥ integrin antibody clone 69-6-5, were obtained from Immunotech. The anti-αᵥβ₃ integrin antibody clone LM609, anti-αᵥβ₆ integrin antibody clone 10D5, and anti-αᵥβ₅ integrin antibody clone P1F6, were from Chemicon. The isotype control (MOPC21) was from Sigma. The αᵥβ₃/αᵥβ₅ inhibitor GW372205X (also called SB223245) was synthesized in house. GW372205X/SB223245 is described in WO-A-96/00730 (SmithKline Beecham Corp) and Keenan et al (1997) J. Med. Chem. 40, 2289-2292. LAP-β₃ was obtained from Sigma, fibrinogen was obtained from Calbiochem, and vitronectin was purified from human plasma The αᵥβ₆ inhibitor GW603365A was synthesized in house and is Ac—RTDLDSLRT-NH₂ (Described in WO 0037487—Merck KgAA). The α4β1 inhibitor BIO1211 was synthesized in house. BIO121 1 is described in Lin et al (1999) J. Med. Chem. 42: 920-934.

GST fusion proteins were made by oligonucleotide insertion into the pGEX-2T vector (Amersham Pharmacia Biotech) using the BamHI and EcoRI restriction sites.

Adhesion Assays

All proteins were coated onto Maxisorp 96-well plates (Nunclon) overnight at 4° C. Each protein was diluted in PBS at the concentrations indicated, and a total volume of 100 ul added per well. Plates were washed twice in PBS then blocked with 3% BSA/PBS for 1 hour at 37° C., and finally washed twice in PBS. Cells were pelleted and washed once in HBSS (Sigma) then spread in HBSS, 25 mM HEPES pH7.5 at the desired cell concentration (K562-WT, K562-αᵥβ₆, at 3×10⁶ cells/ml, K562-αᵥβ₅, K562-α_{vβ}₃ at 2×10⁶ cells/ml, 100 ul per well), in the presence of either 2 mM MgCl₂ or 0.5 mM MnCl₂ as indicated in the figure legends. Other additions are detailed in the figure legends. For antibody inhibition cells were pre-incubated with the antibody (10 μg/ml) on ice for 5 minutes. Cells were then allowed to attach for 35 minutes at 37° C., washed twice with PBS, once in ethanol, and fixed in ethanol for 20 minutes at room temperature. For quantitation cells were visualised by staining with 0.1% crystal violet (Sigma) for 10 minutes then lysed in 0.5% Triton X-100 (Sigma) and read optically at 570 nm in a Wallac Victor plate reader.

### Example 1

### Adhesion of Various Integrins to GST-LAP-β3 Minimal Binding Domain

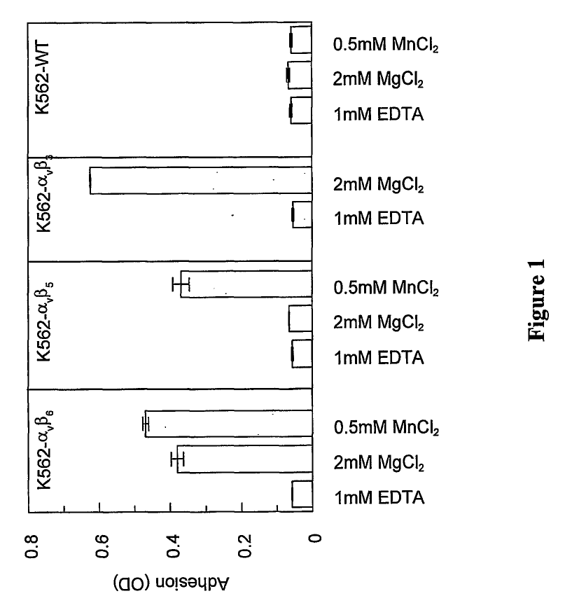

The region of LAPβ₃ corresponding to amino acids 259 to 269 (²⁵⁹HGRGDLGRLKK²⁶⁹) was expressed as a GST fusion protein and tested for the ability to support adhesion of K562-αᵥβ₃, K562-αᵥβ₅, and K562-αᵥβ₆ cells. The GST-LAPβ₃ (amino acids 259-269) protein supported adhesion of K562-αᵥβ₃ and K562-αᵥβ₆ cells in the presence of magnesium, whilst K562-αᵥβ₅ cells required manganese super-stimulation, and K562-WT cells failed to adhere in either cation condition (FIG. 1).

### Example 2

### Specificity of K562-αᵥβ₃, K562-αᵥβ₅, and K562-αᵥβ₆ Cell Binding to GST-LAP-β3 Minimal Binding Domain

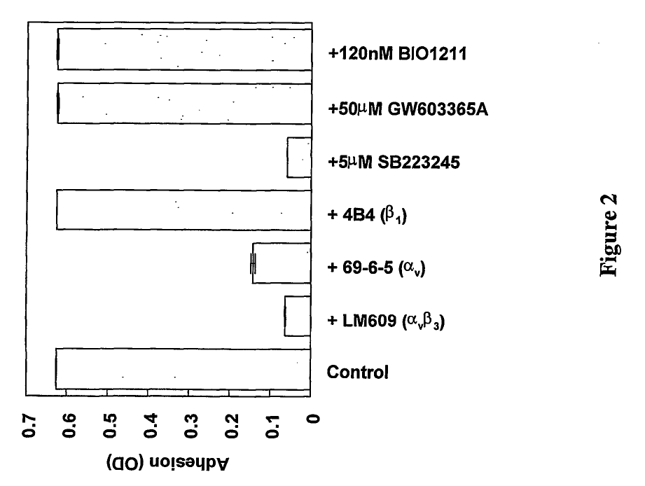

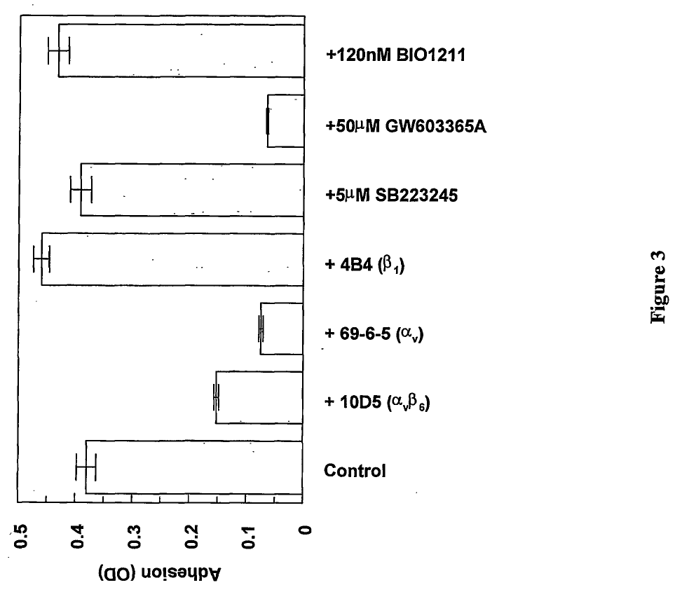

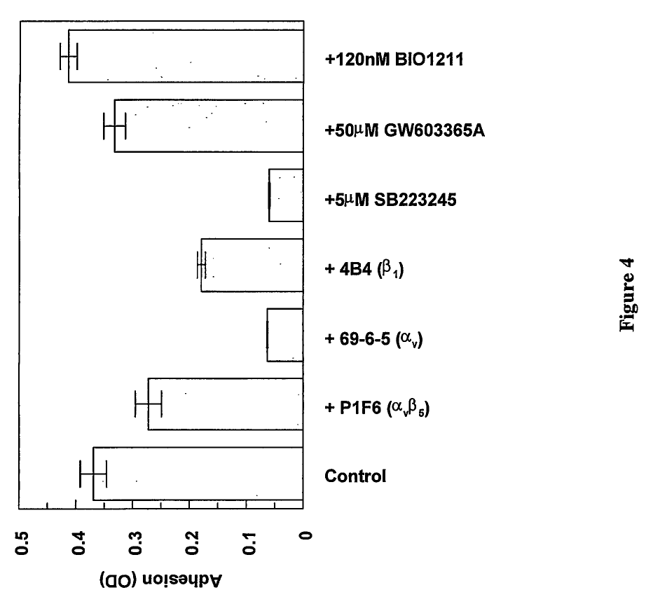

The specificity of the observed adhesion of K562-αᵥβ₃, K562-αᵥβ₅, and K562-αᵥβ₆ cells to LAPβ₃ was confirmed by using specific integrin blocking antibodies. K562-αᵥβ₃ cell adhesion was blocked by antibodies to αᵥβ₃ and αᵥ and the αᵥβ₃/αᵥβ₅ inhibitor SB223245 (FIG. 2). K562-αᵥβ₆, cell adhesion was blocked by antibodies to αᵥβ₆ and αᵥ, and the αᵥβ₆ peptide inhibitor GW603365A (FIG. 3). K562-αᵥβ₅ cell adhesion was completely inhibited by the αᵥ blocking antibody, and partially inhibited by antibodies to αᵥβ₅ and β₁, with total blockade also observed by SB223245 (FIG. 4). Collectively this data confirms that K562-αᵥβ₃ cells adhere to LAPβ₃ via αᵥβ₃, K562-αᵥβ₆ adhere via αᵥβ₆, and K562-αᵥβ₅ adhere vi a mixture of αᵥβ₅ and αᵥβ₆ integrins, as observed for adhesion to LAPβ₁.

### Example 3

### Specificity of K562-αᵥβ₃, K562-αᵥβ₅, and K562-αᵥβ₆ Cells adhere to GST-LAP-β3 Minimal Binding Domain via the RGD motif

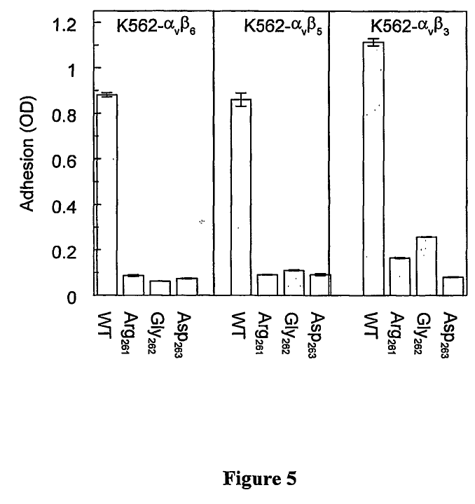

To assess the importance of the RGD motif in LAPβ₃ for integrin binding, fusion proteins were made of GST-LAPβ₃ (amino acids 259-269) where Arg₂₆₁, Gly₂₆₂, or Asp₂₆₃ were separately exchanged for alanine. Analysis of integrin binding properties demonstrated that mutation of any of these residues abolished binding of K562-αᵥβ₃, K562-αᵥβ₆, and K562-αᵥβ₅ cells (FIG. 5). This clearly shows that αᵥβ₁, αᵥβ₃, αᵥβ₅, and αᵥβ₆ all interact with LAPβ₃ via the RGD motif as predicted.

1. A method for the identification of a modulator of the interaction between latency associated peptide LAP of transforming growth factor-β3 (TGF-β3) and an αᵥ integrin which method comprises:
(a) providing, as a first component, latency-associated peptide-β3 (LAP-β3) or a functional variant thereof;
(b) providing, as a second component, an αᵥ integrin thereof or a functional variant thereof;
(c) contacting the two components with a test product under conditions that, in the absence of the test product, would permit the two components to interact; and
(d) determining whether the test product is capable of modulating the interaction between the first and second components, thereby to determine whether the test product is a modulator of the interaction between LAP-β3 and the αᵥ integrin.

2. A method according to claim 1, wherein in step (a) LAP-β3 or a functional variant thereof is in the form of a monomer.

3. A method according to claim 1, wherein in step (a) LAP-β3 or a functional variant thereof is in the form of a dimer.

4. A method according to claim 1, wherein in step (a) LAP-β3 or a functional variant thereof is provided as a component of a small latency complex (SLC).

5. A method according to claim 1, wherein in step (a) LAP-β3 or a functional variant thereof is provided as a component of a large latency complex (LLC).

6. A method according to claim 1, wherein LAP-β3 or a functional variant thereof is fused to a carrier polypeptide.

7. A method according to claim 1, wherein in step (b) the integrin is provided in the form of a cell line which expresses said integrin.

8. A method according to claim 1, wherein the modulator is an inhibitor of the interaction between LAP-β3 and the αᵥ integrin.

9. A method according to claim 1, wherein the modulator is an activator of the interaction between LAP-β3 and the αᵥ integrin.

10. A test kit for the identification of a modulator of the interaction between LAP-β3 and an αᵥ integrin, which kit comprises:
(i) a first component as defined in step (i) of claim 1; and
(ii) a second component as defined in step (ii) of claim 1.

11. A test kit according to claim 10, which kit further comprises:
(iii) means for determining whether a test product modulates the interaction between the first component and the second component.

12. A modulator of the interaction between LAP-β3 and an αᵥ integrin.

13. A modulator according to claim 12 which is an inhibitor of the interaction between LAP-β3 and the integrin.

14. A modulator according to claim 12 which is an activator of the interaction between LAP-β3 and the integrin.

15. A modulator according to claim 13 for use in a method of immuno-modulation or in a method of treatment an inflammatory disease, a fibrotic disease, cancer, diabetic retinopathy, bone resorption or osteoporosis.

16. A modulator according to claim 14 for use in a method of preventing apoptosis.

17-18. (Cancelled)

19. A pharmaceutical composition comprising a pharmaceutically acceptable carrier or diluent and, as active ingredient, a product which is a modulator of the interaction between LAP-β3 and an αᵥ integrin.

20. A method for treating a host requiring immuno-modulation or suffering from an inflammatory disease, a fibrotic disease, cancer, diabetic retinopathy, bone resorption or osteoporosis which method comprises administering to the host an effective amount of a product which is an inhibitor of the interaction between LAP-β3 and an αᵥ integrin.

21. A method for treating a host requiring prevention of apoptosis which method comprises administering to the host an effective amount of an activator of the interaction between LAP-β3 and an integrin αᵥ integrin.

22. A modulator of the interaction between LAP-∃3 and an αᵥ integrin identified by a method according to claim 1.

23. A modulator according to claim 22 which is an inhibitor of the interaction between LAP-∃3 and the integrin.

24. A modulator according to claim 22 which is an activator of the interaction between LAP-∃3 and the integrin.

25-29. (Cancelled)

30. A pharmaceutical composition comprising a pharmaceutically acceptable carrier or diluent and, as active ingredient, a modulator according to claim 22.

31. A method for treating a host requiring immuno-modulation or suffering from an inflammatory disease, a fibrotic disease, cancer, diabetic retinopathy, bone resorption or osteoporosis, which method comprises administering to the host an effective amount of a modulator according to claim 23.

32. A method for treating a host requiring prevention of apoptosis, which method comprises administering to the host an effective amount of a modulator according to claim 24.

33. A method for identifying a product which can be used in a method of immuno-modulation or in a method of treatment of an inflammatory disease, a fibrotic disease, cancer, diabetic retinopathy, bone resorption or osteoporosis, which method comprises:
(a) providing, as a first component, latency-associated peptide-β3 (LAP-β3) or a functional variant thereof;
(b) providing, as a second component, an αᵥ integrin or a functional variant thereof;
(c) contacting the two components with a test product under conditions that, in the absence of the test product, would permit the two components to interact;
(d) determining whether the test product is capable of inhibiting the interaction between the first and second components; and
(e) determining whether a product identified in step (d) can be used in a method of immuno-modulation or in a method of treatment of an inflammatory disease, a fibrotic disease, cancer, diabetic retinopathy, bone resorption or osteoporosis.

34. A method for identifying a product which can be used in a method of preventing apoptosis, which method comprises:
(a) providing, as a first component, latency-associated peptide-β3 (LAP-β3) or a functional variant thereof;
(b) providing, as a second component, an αᵥ integrin or a functional variant of any of these integrins;
(c) contacting the two components with a test product under conditions that, in the absence of the test product, would permit the two components to interact;
(d) determining whether the test product is capable of activating the interaction between the first and second components; and
(e) determining whether a product identified in step (d) can be used in a method of preventing apoptosis.

35. A method for treating a host requiring immuno-modulation or suffering from an inflammatory disease, a fibrotic disease, cancer, diabetic retinopathy, bone resorption or osteoporosis, which method comprises:
(a) identifying a product by use of a method according to claim 33;
and (b) administering to the host an effective amount of the product.

36. A method for treating a host requiring prevention of apoptosis, which method comprises:
(a) identifying a product by use of a method according to claim 34;
and (b) administering to the host an effective amount of the product.

37. (Cancelled)


SEQUENCE LISTING (1 sequence)

SEQ ID NO: 1 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS